(19) United States
(12) Patent Application Publication
(10) Pub. No.: US 2021/0073719 A1
(43) Pub. Date: Mar. 11, 2021

(54) VISUAL INTERACTIVE APPLICATION FOR SAFETY STOCK MODELING

(71) Applicant: Becton, Dickinson and Company, Franklin Lakes, NJ (US)

(72) Inventors: Karen Byron, Ringwood, NJ (US); Dipti Madhani, Bridgewater, NJ (US); Thomas Long, Oakland, NJ (US)

(21) Appl. No.: 16/771,896

(22) PCT Filed: Feb. 20, 2019
(86) PCT No.: PCT/US2019/018693

§ 371 Date: Jun. 11, 2020

Related U.S. Application Data
(60) Provisional application No. 62/635,078, filed on Feb. 26, 2018.

(51) Int. Cl. G06Q 10/08; G06F 16/901; G06F 16/9035

(52) U.S. Cl. CPC G06Q 10/087; G06F 16/9035; G06F 16/9024; G06F 16/9017

(57) ABSTRACT

A safety stock modeling (“SSM”) system is disclosed herein. The SSM system can provide users with the ability to visualize demand data and lead time data at the same time. The SSM system can include a user interface which allows the user to visualize data and manipulate the data. The SSM system provides a safety stock recommendation, which can be updated as the user manipulates the data. The SSM system can allow the user to visualize extreme values or outliers in the data, which can be excluded from the safety stock recommendation.

### RELATED U.S. APPLICATIONS

This application claims priority to U.S. Provisional Appl. No. 62/635,078 filed on Feb. 26, 2018, which is hereby incorporated by reference in its entirety.

### BACKGROUND

### Field

This disclosure relates to systems and methods for safety stock modeling, and, in particular, to an integrated system to output a safety stock recommendation that includes a user interface designed to allow the user to manipulate one or more data points.

### Description

Safety stock is the extra units of stock to mitigate the risk of a shortfall due to uncertainty in supply and demand. As one example, safety stock helps to mitigate the risk on the supply side that a supplier is unable to deliver on time. As another example, safety stock helps to mitigate the risk on the demand side that sales are different than forecasted.

Management of safety stock may include uploading raw data using a template or direct data feed. The output can be a report, such as a spreadsheet. In a complex supply chain, the raw data can include many thousands of data points. Various factors can contribute to safety stock modeling including demand, source lead time, and service level. The demand is the units purchased by end users. The source lead time is the delay between the date of reorder and the date of availability, typically calculated as a number of days. The service level is the probability of meeting demand during the source lead time without a shortfall. There are many other factors which may impact the safety stock recommendation.

One aspect of effective safety stock modeling is allowing a company to understand large quantities of data to effectively manage the safety stock. Unfortunately, many planning systems offer an incomplete picture of how both the supply and the demand impact a safety stock recommendation. To improve safety stock modeling, it would be beneficial if a company could understand how each data point impacts the safety stock recommendation, as well as how changes in certain inputs, such as the service level or the demand forecast, can impact the safety stock recommendation.

### SUMMARY

A system for providing safety stock modeling is disclosed herein. The safety stock modeling can provide a user with an intuitive and interactive interface to manipulate various factors that impact a safety stock recommendation.

In one aspect, a system for providing safety stock modeling for a medical product is provided. In one non-limiting example, the system can include a database of information related to a plurality of medical products. The system can include an engine comprising a demand module, a sourcing lead time module, and at least one analytic module for calculating a trend in data points from the database of information. The system can include a user interface designed to allow the user to manipulate an interactive graphical display, wherein the interactive graphical display illustrates data points from the database of information related to a single product in a demand coordinate system and a sourcing lead time coordinate system. The engine can be designed to output an optimal safety stock recommendation based on included data points in the demand coordinate system and included data points in the sourcing lead time coordinate system. The engine can be designed to update the optimal safety stock recommendation as the user changes the included data points.

In some embodiments, the database of information comprises data over at least two years. In some embodiments, the engine is designed adjust the optimal safety stock recommendation based on the trend calculated by the at least one analytic module. In some embodiments, the trend is a cyclic demand. In some embodiments, the interactive graphical display is updated in real-time when the user changes the included data points. In some embodiments, the interactive graphical display provides information from the database of information on a selected data point. In some embodiments, the engine is designed to provide a demand threshold in the demand coordinate system and a sourcing threshold in the sourcing lead time coordinate system. In some embodiments, the optimal safety stock recommendation is updated when the user excludes one or more data points above the demand threshold or the sourcing threshold. In some embodiments, the optimal safety stock recommendation is updated when the engine excludes one or more data points above the demand threshold or the sourcing threshold. In some embodiments, the engine is designed to create a report from the included data points. In some embodiments, the optimal safety stock recommendation is updated when the service level is adjusted. In some embodiments, the interactive graphical display is designed to allow the user to visualized data points in the demand coordinate system and the sourcing lead time coordinate system simultaneously. In some embodiments, the user interface is designed to allow the user to interactively exclude data points in the demand coordinate system and the sourcing lead time coordinate system simultaneously. In some embodiments, the user interface is designed to allow the user to interactively exclude data points in the demand coordinate system and the sourcing lead time coordinate system independently. In some embodiments, the engine is designed to output the optimal safety stock recommendation based on the mean and standard deviation of the included data points. In some embodiments, the engine is designed to output an optimal safety stock recommendation for a family of related medical products.

In one aspect, a method for providing safety stock modeling for a medical product is provided. The method can include storing information related to a plurality of medical products in a database. The method can include generating with an engine an interactive graphical display illustrating data points related to demand and sourcing lead time. The method can include updating, in real time, the interactive graphical display and an optimal safety stock recommendation by adjusting an input selected from the group consisting of a trend of data points, a user excluded data point, a user included data point, and an engine excluded data point. In some embodiments, the input is a user excluded data point, wherein the user excluded data point is above a demand threshold or a sourcing threshold. In some embodiments, the input is an engine excluded data point, wherein the engine excluded data point is above a demand threshold or a sourcing threshold. In some embodiments, the method can include updating, in real time, the interactive graphical display and the optimal safety stock recommendation based on service level.

These and other features of this disclosure will become more fully apparent from the following description.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 is a block diagram illustrative of a main system according to one embodiment.

FIG. 2 is a flowchart illustrating an example of a safety stock modeling (“SSM”) system of FIG. 1.

FIG. 3 is a flowchart illustrating an example of a method of generating demand and supplier graphs of the SSM system of FIG. 2.

FIG. 4 is a flowchart illustrating another example of a method of generating demand and supplier graphs utilizing forecast data of the SSM system of FIG. 2.

FIG. 5 is a flowchart illustrating an example of a method of adjusting the demand and supplier graphs of the SSM system of FIG. 2.

FIG. 6A is a screenshot illustrating an example of a demand graph of the SSM system of FIG. 2.

FIG. 6B is a screenshot illustrating an example of a supplier graph of the SSM system of FIG. 2.

FIG. 7 is a screenshot illustrating an example of an interactive graphical display of the SSM system of FIG. 2.

FIGS. 8A-9B are screenshots illustrating examples of excluded data points of example demand and supplier graphs of the SSM system of FIG. 2.

FIG. 10 is a screenshot illustrating an example of additional information generated from a data point of the SSM system of FIG. 2.

FIG. 11 is a screenshot illustrating an example of a report of the SSM system of FIG. 2.

### DETAILED DESCRIPTION

A safety stock modeling (“SSM”) system is described herein. As will be appreciated by one skilled in the art, there are numerous ways of carrying out the examples, improvements, and arrangements of the SSM system in accordance with embodiments of the present invention disclosed herein. Although reference will be made to the illustrative embodiments depicted in the drawings and the following descriptions, the embodiments disclosed herein are not meant to be exhaustive of the various alternative designs and embodiments that are encompassed by the disclosed invention, and those skilled in the art will readily appreciate that various modifications may be made, and various combinations can be made, without departing from the invention.

Although described throughout this application as a safety stock modeling system, this disclosure is not intended to be limited only thereto. For example, the system can be modified for use in modeling other data, such as big data, that may be analyzed to reveal trends and outliers, especially related to business or business interactions. For ease of description, however, the system will be described with reference to safety stock modeling as an illustrative example. However, the embodiments are not only limited to safety stock modeling systems and methods.

The SSM system can utilize many types of data, including, but not limited to, demand data, supplier data, and forecast data. As one non-limiting example, demand data can include the number of units sold and the date of sale. As another non-limiting example, supplier data can include source lead time data including days until completion and completion date. As yet another non-limiting example, forecast data can include a trend of the demand data, such as a seasonal trend, a cyclic trend, an upward trend, or a downward trend. Further, this disclosure refers to demand data, forecast data, and supplier data. This is done in the context of a non-limiting example. The SSM system can include data related to one or more additional persons or entities, including one or more suppliers, one or more distributors, and/or one or more end users, without limit. In some embodiments, not all types of data will be available for a given product. For instance, as one non-limiting example, forecast data may not be available for a product. The SSM system can be utilized for one or more types of data. For ease of description, this disclosure describes the SSM system with reference to data and data points. Reference to “data” is intended to encompass all types of data.

The SSM system can be used by many types of people, including those who analyze and predict safety stock for one or more products. The user can be any person who wishes to understand improvement opportunities within the demand data and the supplier data. The SSM system can be utilized by one or more suppliers to understand supplier data. The SSM system can be utilized by one or more distributors to understand demand data or forecast data. The SSM system can be utilized by a user to understand the influence of both demand data and supplier data on the safety stock recommendation. The SSM system can be utilized by one or more users, which can be any person, without limit, with access to the SSM system.

As described herein, the SSM system can allow the user to visualize demand data and supplier data together, and in some embodiments, demand data, forecast data, and supplier data together. In some embodiments, the SSM system allows a user to visualize demand data, with or without trend adjustment. For instance, the SSM system can provide an interactive graphical display that illustrates demand data, which can also include forecast data related to the demand data. The interactive graphical display can also illustrate supplier data. The SSM system can allow a user to visualize both demand variability and supplier variability together. Unlike other systems, the interactive graphical display can illustrate both the demand data and the supplier data within the same visual space, such as in a side-by-side format. The interactive graphical display provides an intuitive, easy to understand format the demand data and supplier data.

The SSM system can give a user the ability to visualize outliers in demand data and supplier data. The SSM system can allow a user to quickly identify outliers. The user can decide whether one or more outliers should be removed or whether one or more outliers should be included as true process variability. The SSM system can allow the user to remove extreme values for both demand data and supplier data. In some embodiments, the SSM system can allow a user to simultaneously remove extreme values for both demand data and supplier data. In some embodiments, the SSM system can allow a user to independently remove extreme values for both demand data and supplier data. The SSM system can allow the user to reversibly include or exclude one or more data points for the calculation of a safety stock recommendation. The user can gain an understanding of one or more outliers by understanding their impact on the safety stock recommendation.

The SSM system can give a user the ability to gain a greater understanding of an individual data point. In some embodiments, selecting a data point, such as by hovering over the data point, can provide additional information related to the data point. The additional information of the data point can be viewed on the interactive graphical display of the user interface. The additional information related to the data point can allow the user to understand the root cause of the outlier. In some embodiments, the additional information enables a user to perform root cause analysis to identify the root cause of the outlier. The SSM system can give a user the ability to gain a greater understanding of a group of data points. The SSM system can give a user the ability to gain an understanding of a family of related products, such as one or more families of medical products. In some embodiments, the SSM system can provide an overview report for families of product.

As will become apparent from the following description, the SSM system can allow manipulation of the demand data and supplier data. The SSM system can provide the ability to model the impact of what-if scenarios by allowing a user to interact with the interactive graphical display. The interactive graphical display can also allow a user to remove one or more data points from the safety stock calculations. The interactive graphical display can provide additional information regarding the one or more data points, which may allow the user to gain a better understanding of extreme values. In some embodiments, the SSM system can allow a user to visualize the results of changing variables in real-time. The SSM system can give a user the ability to visualize the impact of what-if scenarios.

In some embodiments, the user can change confidence level, model type, and/or remove extreme values. As one example, an extreme value can be unrepresentative such that the user chooses to exclude the extreme values. As another example, an extreme value can be beyond a threshold which can be calculated by the SSM system. In some embodiments, one or more data points can be removed from the calculations for the safety stock recommendation by the user selecting points or regions for removal. In some embodiments, one or more data points can be removed from the calculations for the safety stock recommendation by applying an auto-removal function.

The SSM system can provide a visual or graphical illustration of data. In some embodiments, the SSM system can generate scatter plots. The SSM system can generate a demand scatter plot, with or without trend data. The SSM system can generate a supplier scatter plot. A scatter plot is a mathematical diagram that uses Cartesian coordinates to display data points related to two variables. Typically, the two variables form the axes of the diagram. As one example, for the demand scatter plot, the two variables can be the number of units sold and the date of sale. As another example, for the supplier scatter plot, the two variables can be days to completion and date of completion. Other variables for the scatter plots are contemplated. The scatter plots can allow the user to visualize process variability. The scatter plots can allow the user to easily visualize an outlier on the scatter plot. The user can exclude the outlier directly by interacting with the scatter plot to visualize an improvement opportunity. The SSM system can allow a user to visualize and distinguish a product with a skewed safety stock recommendation. For instance, a product may have a skewed safety stock recommendation due to a small number of outliers which are easily identifiable on the scatter plot.

In some embodiments, each data point can be represented on the scatter plot as an icon. As described herein, the user can decide to exclude extreme values to make the safety stock recommendation more representative. In some embodiments, one or more data points that are included in the safety stock calculations have a different icon than one or more data points that are excluded. For example, included data points can be a solid shape, while excluded data points can be the outline of the shape. In some embodiments, the SSM system can allow the user to visualize which data points are included an excluded. In some embodiments, the one or more data points that are excluded remain visible for the user's reference. The SSM system can allow a user to visualize various what-if scenarios by allowing the user to have the ability to reversibly include and exclude one or more data points.

The SSM system can be designed to output a safety stock recommendation. The safety stock recommendation can be based on the demand data and the supplier data. In some embodiments, the stock recommendation can be based on the demand data, the forecast data, and the supplier data. The safety stock recommendation can be displayed on the interactive graphical display. The safety stock recommendation can be displayed as the number of units and/or the number of days. The safety stock recommendation can be a workable output based on the demand data and the supplier data.

The safety stock recommendation can be dynamically updated as conditions are changed. The safety stock recommendation can change as the user includes one or more data points, excludes one or more data points, or alters one or more inputs. Based on the user's manipulation of data, the SSM system can recalculate the safety stock recommendation. In some embodiments, the SSM system can recalculate the safety stock recommendation when the user changes the service level. In some embodiments, the SSM system can recalculate the safety stock recommendation when the user removes one or more extreme values. As described herein, the SSM system can be designed to output a safety stock recommendation in response to a user manipulating the data. In some embodiments, the safety stock recommendation can be provided on the interactive graphical display and updated in real-time as the user manipulates the data. Therefore, the changes to the safety stock recommendation become more intuitive as the user manipulates the data.

The SSM system can allow the user to gain a better understanding of data, including the demand data, forecast data, and the supplier data. The SSM system can present the data as a graph, in some embodiments, a scatter plot. The user can select a data point to be provided with additional information regarding the data point. The user can change one or more inputs such as service level or model type to understand how these inputs impact the safety stock recommendation. The user can remove one or more data points, such as extreme valves that seem unrepresentative. The user can easily visualize data points which are outliers, and in some embodiments, further investigate the root cause of such outliers. The SSM system can provide an overview report related to the data, such as a report of the data included in the safety stock calculations. The report can represent a single product, one or more products, or one or more families of products. The SSM system can be designed to allow the user to understand the influence of each data point of demand data and supplier data. The SSM system can be designed to allow the user to gain insight into the contributions to the safety stock recommendation.

In some embodiments, the SSM system includes a user interface that provides a means for the user to interact with data. The user interface can be any device which enables visual display and interaction by the user including a touchscreen, smartphone, tablet, laptop, computer, or other type of device. The user interface can be connected to a larger network, such as the internet or the cloud, which can provide one or more components of the SSM system described herein such as a database or an engine. The user interface can include an interactive graphical display that can provide a visual display of data, such as one or more graphs. The interactive graphical display can change as the user manipulates data. The interactive graphical display can also change as the user interacts with one or more inputs, such as the service level. The inputs can be entered by the user, such as by typing, touching, or clicking.

In some embodiments, the SSM system includes a database of information. The SSM system can provide an interface to interact with the database. The database can include the demand data and the supplier data. The SSM system can access the database to calculate the safety stock recommendation. The database can store any data described herein. The database can store information related to each data point. The database can store information in any format, including a spreadsheet or other ledger. In the illustrative embodiment, the SSM system can access the database in order to gather, calculate, and analyze data to be presented to the user via the user interface. In some embodiments, the SSM system can access the database to generate a report.

The SSM system can allow the user to interact with the user interface rather than the raw data contained within the database. The SSM system can present the data to the user in an easy to understand format. The SSM system can generate one or more graphs such as one or more scatter plots which provide a user with an intuitive understanding of the variability in the data. In some cases, the user can understand and distinguish the results presented in an intuitive graph better than data presented in a spreadsheet. The user can distinguish whether the results are skewed by one or more outliers when presented as a graph. In some embodiments, a user can quickly correlate an outlier with the underlying raw data, such as by hovering or selecting a data point on the graph. The SSM system can reduce the time consuming efforts to understand the raw data, such as by manually reviewing the raw data. The SSM system can reduce the time consuming efforts to manipulate the raw data, such as by manually changing the service level. In the supply chain world, raw data can be several thousand, or million, data points. The SSM system can provide the user with the ability to visualize all of the data points, or a portion thereof, at once.

As described herein, the raw data can be a large amount of data. The size of the data can prevent the user from being able to create what-if scenarios without time consuming manual effort. The SSM system can provide can allow the user to reversibly test what-if scenarios. The SSM system can provide instant results to the user when the user manipulates one or more inputs. The SSM system can allow the user to understand how different variables impact the safety stock recommendation without spending time to change the raw data set. The SSM system can quickly include or exclude data points from the calculations. The SSM system can calculate and recalculate the safety stock recommendation based on the user's interactions with the SSM system. The SSM system can provide the user with the ability to visualize extreme values, allowing for quick insight into the supply chain process. The SSM system can be designed to be user friendly and allow interactive modeling at the user's fingertips.

The SSM system can allow multiple iterations or recalculations during a method of use. As described herein, the SSM system can collect demand data and supplier data. The SSM system can provide a graphical representation of the demand data and the supplier data. The SSM system can calculate the recommended safety stock. The SSM system can generate a report based on the demand data and supplier data. In some uses, the user can manipulate the data. In some embodiments, the SSM system can collect the selected or included demand data and the selected or included supplier data. The SSM system can provide a graphical representation of the selected or included demand data and the selected or included supplier data. The SSM system can recalculate the recommended safety stock. The SSM system can regenerate a report based on the demand data and supplier data. The SSM system can repeat one or more steps for scenarios chosen by the user interacting with the SSM system.

The SSM system can help with balancing the risk of stock-outs and the risk of carrying excess inventory. The SSM system, including the data graphs and the interactive capabilities, can provide a useful demonstration tool to audiences both internal and external to the company. The SSM system can provide graphs for each product to demonstrate why certain products require increased inventory versus others. The SSM system can provide interactive capabilities to understand each product and the corresponding safety stock recommendation. The SSM system can provide a safety stock recommendation for a product or one or more families of products.

In the following description, specific details are given to provide a thorough understanding of the examples. However, it will be understood by one of ordinary skill in the art that the examples may be practiced without these specific details. For example, components/devices may be shown in block diagrams in order not to obscure the examples in unnecessary detail. It is also noted that the examples may be described as a process, which is depicted as a flowchart, a flow diagram, or a block diagram. Although a flowchart may describe the operations as a sequential process, many of the operations can be performed in parallel, or concurrently, and the process can be repeated. In addition, the order of the operations may be re-arranged. A process is terminated when its operations are completed. A process may correspond to a method, a function, a procedure, a subroutine, a subprogram, etc. When a process corresponds to a software function, its termination corresponds to a return of the function to the calling function or the main function.

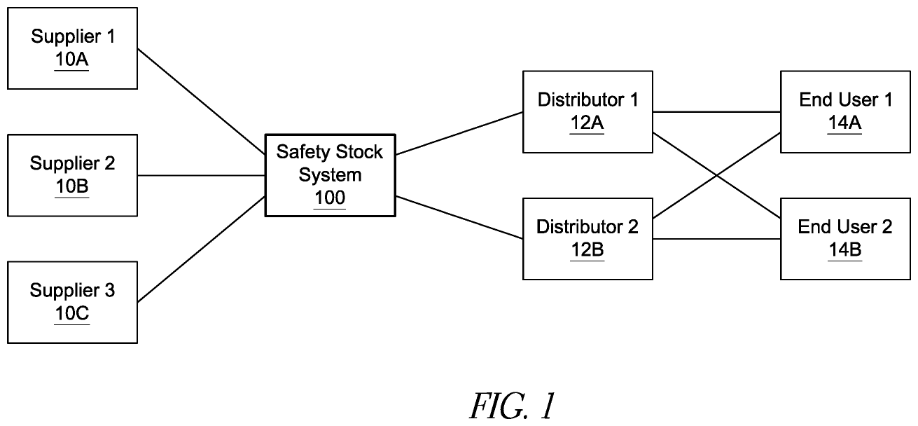

FIG. 1 is a block diagram that includes a safety stock modeling (“SSM”) system 100 according to some embodiments. Several additional users can provide data to the SSM system. In the illustrated embodiment of FIG. 1, these additional users include one or more suppliers 10A-C, one or more distributors 12A-B, and one or more end users 14A-B. While three suppliers 10A-C are shown in FIG. 1, any number of suppliers can provide supplier data. Similarly, while two distributors 12A-B and two end users 14A-B are shown in FIG. 1, any number of distributors and/or end users can provide demand data. The SSM system 100 can be utilized by a company, which is the receiver of goods from one or more of the suppliers 10A-C and the deliverer of goods to one or more of the distributors 12A-B. In some embodiments, the SSM system 100 can calculate the safety stock recommendation related to one or more of the company's products. In some embodiments, the product can be a medical product. One or more medical products can be considered a family of medical products. The SSM system 100 can calculate the safety stock recommendation related to a single product, a family of products, or families of products.

In some embodiments, the data related to each supplier can relate to individual transactions. The data related to each supplier can include the number of units delivered to the company. The data related to each supplier can include the number of days until delivery. The data related to each supplier can include the date of delivery. The data related to each supplier can be source lead time data. Other data related to the one or more suppliers can be provided as supplier data.

Similarly, in some embodiments, the data related to each distributor can relate to individual transactions. The data related to each distributor can include the number of units delivered by the company. The data related to each distributor can include the number of days until a sale. The data related to each distributor can include the date of sale. In some embodiments, the data related to each distributor can include data on one or more end users who purchase the product. The data related to each end user can include identifying information regarding the end user, such as name or location. In the illustrated embodiment of FIG. 1, the end user 14A can purchase a product from one or more distributors 12A-B. The data related to each end user can relate to variability in selecting the distributor 12A-B by the end user 14A.

In some embodiments, the data related to the one or more suppliers, distributors, and end users can be collected by the company. The company is the one that utilizes the SSM system 100. In some embodiments, the demand data can be collected by business software. The forecast data, if available, can be collected by business software. The demand and the forecast data can be acquired from SAP® using Demand Planning (DP) Waterfall. The forecast data can be acquired from the SAP® Forecast. Other business software by other providers may be utilized to collect demand and/or forecast data. In some embodiments, the data can be collected once to provide data on recent actuals and forecast. In some embodiments, the data can be collected once to provide longer historical actuals. Therefore, in some embodiments, in order to process demand data, the demand data is collected twice for the SSM system. The demand data can span a period of time. As described herein, the SSM system can calculate trends of demand data. In some embodiments, at least two years of historical data can be collected to evaluate seasonality. In some embodiments, the trend of the demand data can be improved data over a longer period of time is included, for instance over a period of three years, four years, five years, etc. The company and/or the SSM system can process the data to ensure numbers, dashes, dates, and blanks are handled appropriately.

In some embodiments, the data coming from each supplier can be collected by business software. The supplier data can be acquired from the Enterprise Resource Planning system, or ERP system, with MB51 reports. Other business software may be utilized to collect supplier data. The supplier data can include source lead time data. In some embodiments, the source lead time calculations can begin when finished goods enter the system. In some embodiments, the source lead time does not include manufacturing time. In some embodiments, the source lead time can include any delays and transfers, including delays and transfers for one or more processes such as sterilization and delays and transfers to one or more locations such as distribution centers. The supplier data can be collected for specific products, such as specific material number. The supplier data can be span a period of time. The supplier data can span the same period of time as the demand data. One or more of the following fields can be included in the supplier data: material, material description, plant, movement type, movement type text, posting date, and batch. The company and/or the SSM system 100 can process the data to ensure numbers, dashes, dates, and blanks are handled appropriately.

As described herein, the SSM system 100 can calculate the safety stock recommendation related to one or more products, and in some embodiments, one or more medical products. The medical product can be sterilized which is factored into the source lead time. The material number can indicate whether the medical product is sterilized, whether sterilization is complete, or whether the medical product is not sterilized. In some embodiments, only batches which complete their life cycle within a certain time period are included in the source lead time calculations. In some embodiments, a batch completes its life cycle based on movement within a plant location and/or distributor center location. In some embodiments, a batch does not include further shipping among distribution centers. The locations can be classified by the SSM system and/or the company. In some embodiments, how locations are classified as plants or distribution centers is specific to each business unit within the company.

In some embodiments, the source lead time is calculated as the last day a batch arrives in a given distribution center minus the first day it is entered at the first plant. In some embodiments, the source lead time is calculated as the duration between the entry of the batch at any plant and the entry of the batch at a given distribution center. In some embodiments, the source lead time is calculated as the sum of lead time when the product is not sterilized and the lead time when the product is sterilized. In some embodiments, the sterilization can begin prior to the arrival of the complete batch which can lead to an overstatement of the lead time. In some embodiments, there is a delay after sterilization and prior to shipping to a distribution center which can lead to an understatement of the lead time. In some embodiments, the source lead time is calculated based on the first versus last days at both the plant and the distribution center. In some embodiments, the lead time is calculated without reference to the path taken by the batch. In some embodiments, the source lead time is calculated differently for individual products than a kit. In some embodiments, a finished kit can include other finished goods, which may or may not be calculated using the SSM system . In some embodiments, the lead time for one or more finished goods of a kit is excluded from the calculations of the SSM system.

In some embodiments, the source lead time can be calculated in days. In some embodiments, the source lead time can be calculated in days and converted to one or more other units of time, such as months. In some embodiments, the demand data and supplier data is converted to the same unit of time for the safety stock calculations.

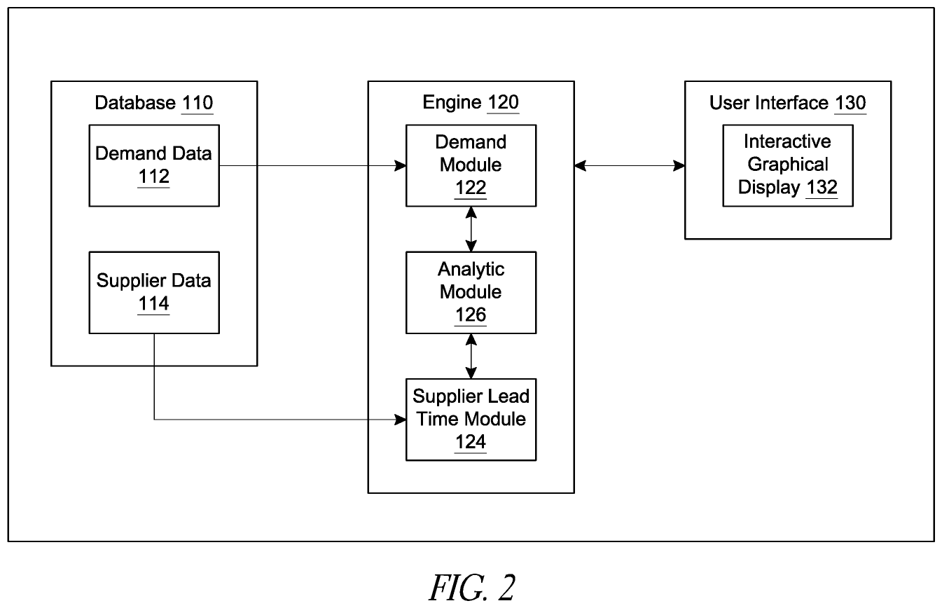

FIG. 2 is a block diagram of the SSM system 100 according to one embodiment. In the illustrative embodiment shown in FIG. 2, the SSM system 100 includes a database 110, an engine 120, and a user interface 130. In some embodiments, one or more of these components can be omitted. In some embodiments, the SSM system 100 contains additional components not shown in FIG. 2. The SSM system 100 can be embodied in a single device (e.g., a single computer or server) or distributed across a plurality of devices (e.g., a plurality of computers or servers). The modules or elements of the SSM system 100 can be embodied in hardware, software, or a combination thereof.

The modules or elements may comprise instructions stored in one or more memories and executed by one or more processors. Each memory can be a RAM memory, a flash memory, a ROM memory, an EPROM memory, an EEPROM memory, a register, a hard disk, a removable disk, a CD-ROM, or any other form of storage medium known in the art. Each of the processors may be a central processing unit (CPU) or other type of hardware processor, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or in the alternative, the processor may be any conventional processor, controller, microcontroller, or state machine. A processor may also be implemented as a combination of computing devices, for example, a combination of a DSP and a microprocessor, a plurality of microprocessors, one or more microprocessors in conjunction with a DSP core, or any other such configuration. Exemplary memories are coupled to the processors such that the processors can read information from and write information to the memories. In some embodiments, the memories may be integral to the processors. The memories can store an operating system that provides computer program instructions for use by the processors or other elements included in the system in the general administration and operation of the SSM system 100.

The database 110 can be a database of information, such as a database of raw data. The database 110 can include information related to a single product, such as a single medical product. The database 110 can include information related to two or more products, such as a plurality of medical products. The database 110 can include information related to a family or a plurality of families of medical products. The database 110 can comprise a single database or a plurality of databases. In an exemplary embodiment, the SSM system 100 can include one or more databases including demand data 112 and supplier data 114. The data related to each distributor can be stored as demand data 112 in the database 110. The database 110 can also store data related to the end user, if available, separately or as part of the demand data 112. The data related to each supplier can be stored as supplier data 114 in the database 110. In some embodiments, the database 110 can store raw data. In some embodiments, the database 110 can store data that has been processed, such as by software to provide standard formatting. In some embodiments, the database 110 can store data that has been processed, such as by software to remove errors. The database 110 can include data over a period of time. The database 110 can include data over 3 months, 6 months, 9 months, 12 months, 18 months, 24 months, 30 months, 36 months, 42 months, 48 months, or any range of any two of the foregoing values.

The database 110 can store additional information, such as information related to the functionality of the SSM system 100. The database 110 can store one or more reports generated by the SSM system 100. The database 110 can store any information relevant to the SSM system 100 for any calculations, past, present, or future. The database 110 can store data generated during a user's previous interactions with the SSM system 100. This can include the safety stock recommendation, the report, and any manipulations by the user of the data. The SSM system 100 can automatically, or as directed by the user, store data related to the user's interactions with the SSM system 100. In some embodiments, the SSM system 100 can customize future interactions between the SSM system 100 and the user based on past interactions.

The engine 120 can include a demand module 122, a supplier lead time module 124, and an analytic module 126. In some embodiments, the engine 120 can comprise at least one analytic module 126, for instance one analytic module, two analytic modules, three analytic modules, etc. In some embodiments, one or more of the modules can be omitted or combined with another module. In some embodiments, the engine 120 can contain additional modules. The engine 120 can process the demand data 112 and the supplier data 114. The engine 120 can retrieve data from the database 110. The engine 120 can provide one or more output to the user interface 130 as described herein.

The engine 120 can include the demand module 122. The demand module 122 can calculate the mean of the demand data 112. The demand module 122 can calculate the standard deviation of the demand data 112. In some embodiments, the demand module 122 can calculate both the mean and the standard deviation of the demand data 112 to calculate the safety stock recommendation. The demand module 122 can calculate the mean and the standard deviation of the demand data 112, with or without forecast data. In some embodiments, the mean and the standard deviation of the demand data 112 are calculated for a period of time. The period of time can be a number of months, for instance, twelve months, or any other period of time including two weeks, one hundred days, six months, eighteen months, twenty four months, or any range of any two of the foregoing values. The mean and the standard deviation of the demand data 112 can be calculated according to any method.

The demand module 122 can calculate the mean and the standard deviation of the demand data 112, with trend adjustment based on the forecast data. In some embodiments, the demand data 112 is modeled. The mean can be defined as the value projected for a period of time. The period of time can be a number of months, for instance, a month, or any other period of time including two weeks, ten days, two months, etc. The mean can be the average expected for the next month. The standard deviation can be defined as the standard deviation for the residuals after adjusting for any trend. The trend adjustment can involve a change from the previous method of calculating the mean and standard deviation. The trend adjustment can be useful for products with a persistent trend. As one non-limiting example, if demand for a product was trending downward, then the mean and standard deviation would produce an artificially high safety stock recommendation. The trend, in this case, may be interpreted as demand variability instead of a downward trend. As another non-limiting example, two data sets may have the same mean and standard deviation adjusted for the trend, with one data set near a consistent value and one data set trending upward. If the trend is ignored, then the standard deviation of the data set trending upward is artificially increased.

As one non-limiting example, an software process that is used to model the demand data 112 evaluates a matrix of options. In some embodiments, the process that is used to model the demand data 112 evaluates Akaike information criterion (AIC) to select the best model based on the relative quality of each model. The model characteristics can include no trend or additive trend, damping to trend or no damping, no seasonality or additive seasonality. Other characteristics are also contemplated.

The engine 120 can include the supplier lead time module 124. The supplier lead time module 124 can calculate the mean of the supplier data 114. The supplier lead time module 124 can calculate the standard deviation of the supplier data 114. The supplier lead time module 124 can calculate both the mean and the standard deviation of the supplier data 114 to calculate the safety stock recommendation.

The mean and the standard deviation of the supplier data 114 can be calculated for a period of time. The period of time can be a number of months, for instance, twelve months, or any other period of time including two weeks, one hundred days, six months, eighteen months, twenty four months, or any range of any two of the foregoing values. In some embodiments, the mean and the standard deviation of the demand data and the supplier data are calculated for the same period of time. In some embodiments, the mean and the standard deviation of the demand data and the supplier data are calculated for twelve months. In some embodiments, the mean and the standard deviation of the demand data and the supplier data are calculated for different periods of time.

The analytic module 126 can perform any type of analysis for the SSM system 100. The analytic module 126 can perform any type of analysis on the output of the demand module 122. The analytic module 126 can perform any type of analysis on the output of the supplier lead time module 124. In some embodiments, the analytic module 126 can calculate a trend in data. In some embodiments, the analytic module 126 can utilize the demand data 112 to create a forecast. In some embodiments, the analytic module 126 can access forecast data. In some embodiments, the engine 120 can be configured adjust the optimal safety stock recommendation based on the trend calculated by the analytic module 126. In some embodiments, the trend is a cyclic demand. In some embodiments, the analytic module 126 calculates the safety stock recommendation based on outputs of the demand module 122 and the supplier lead time module 124. In some embodiments, the analytic module 126 can detect and correct errors.

The analytic module 126 can provide a means to have an interactive experience with the SSM system 100. Instructions connected to, or part of, the analytic module 126 can process inputs by the user. The analytic module 126 can relay the user inputs to the demand module 122 and the supplier lead time module 124. The analytic module 126 can convert a user input into a format to convey the user input to the demand module 122 and the supplier lead time module 124. The analytic module 126 can convert an output of the demand module 122 and the supplier lead time module 124 into a format to covey to the user interface 130. The analytic module 126 can relay an output from the demand module 122 and/or the supplier lead time module 124 to the user interface 130.

In some embodiments, the engine 120, or a module thereof, can use information from the demand module 122 and the supplier lead time module 124 to calculate a safety stock recommendation from demand data 112 and supplier data 114. The engine 120 can be designed to output an optimal safety stock recommendation based on included and excluded data points and/or one or more user inputs. The engine 120 can be designed to output a graphical representation based on included data points in a demand coordinate system and included data points in a sourcing lead time coordinate system. The engine 120 can be designed to update the safety stock recommendation as the user changes the included data points. In some embodiments, the engine 120 can be designed to output the safety stock recommendation based on the mean and standard deviation of the included data points. In some embodiments, the engine 120 can be designed to output the safety stock recommendation for a product, such as a medical product, or a family of products, such as a family of medical products.

The engine 120 can utilize data collected from the demand module 122 to display a demand graph as described herein. The engine 120 can utilize data from the supplier module 124 to display a supplier graph as described herein. The engine 120 can be responsive to the user's interactions to exclude one or more data points related to the demand data 112 or the supplier data 114. For example, based on the user's exclusion, the demand module 122 can recalculate the mean and standard deviation of the demand data 112. For example, based on the user's exclusion, the supplier module 124 can recalculate the mean and standard deviation of the supplier data 114.

The engine 120 can be responsive to any of the user's interactions as described herein. In the illustrative embodiment, the engine 120 can act as a relay between the user interface 130 and the database 110. The engine 120 can enable real-time manipulation of the data in the database 110 by the user's manipulations with the user interface 130. The engine 120 can update the safety stock recommendation when the user changes one or more inputs. For instance, the engine 120 can update the safety stock recommendation when the user adjusts the service level. The engine 120 can be designed to calculate a demand threshold in the demand coordinate system. The engine 120 can be designed to calculate a supplier threshold in the supplier coordinate system. The engine 120 can be designed to update the optimal safety stock recommendation when the user excludes one or more data points. The engine 120 can be designed to update the optimal safety stock recommendation when the user excludes one or more data points outside of the demand threshold or the supplier threshold. The engine 120 can be designed to update the optimal safety stock recommendation when the engine 120 excludes one or more data points outside the demand threshold or the supplier threshold. The engine 120 can be designed to create a report. The report can be created based on the included data points.

The SSM system 100 can include the user interface 130. The user interface 130 can allow the user to interact with the SSM system 100. In some embodiments, the user interface 130 is any device, such as a smartphone, tablet, or computer. The user can enter one or more inputs while using the user interface 130. The user interface 130 can allow the user to type, click, move, draw or otherwise interact with the user interface 130. The user interface 130 can include an interactive graphical display 132. The interactive graphical display 132 can illustrate any output from the engine 120. The interactive graphical display 132 can allow the user to visualize data. The interactive graphical display 132 can allow the user to visualize and manipulate the information that is being displayed on the interactive graphical display 132. The interactive graphical display 132 can update in real-time as the user interacts with the user interface 130. In some embodiments, the interactive graphical display 132 is any visual display, including a monitor, screen, or touchscreen.

The interactive graphical display 132 can illustrate demand data 112 in a demand coordinate system. The interactive graphical display 132 can illustrate supplier data 114 in a source lead time coordinate system. The interactive graphical display 132 can be designed to allow the user to visualize data points in the demand coordinate system and the sourcing lead time coordinate system simultaneously. The interactive graphical display 132 can be designed to allow the user to visualize data points in the demand coordinate system and the sourcing lead time coordinate system independently. The user interface 130 can be designed to allow the user to interactively exclude data points in the demand coordinate system and/or the sourcing lead time coordinate system, include data points in the demand coordinate system and/or the sourcing lead time coordinate system, and/or change one or more inputs. The interactive graphical display 132 can update in real-time when the user excludes one or more data points, includes one or more data points, or alters an input.

In some embodiments, SSM system 100 can be utilized in a method for providing safety stock modeling for a product, such as a medical product. The method can include storing information related to one or more medical products in the database 110. The method can include generating with the engine 120 an interactive graphical display 132 illustrating data points related to demand and sourcing lead time. The method can include updating, in real time, the interactive graphical display 132 and an optimal safety stock recommendation by adjusting an input selected from the group including a trend of data points, a user excluded data point, a user included data point, and an engine excluded data point. Other methods involving the SSM system 100 are described herein.

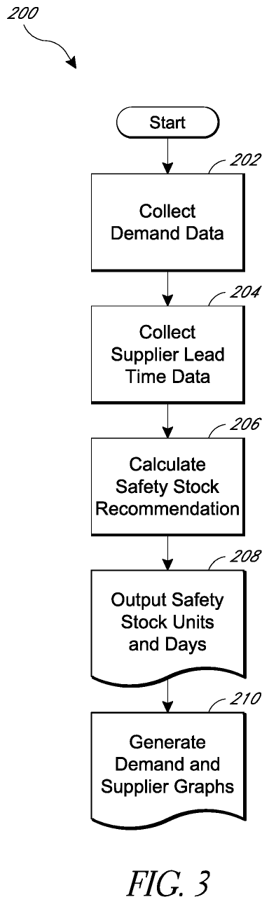

FIG. 3 is a flowchart illustrating an example process 200 of the engine 120 of the SSM system 100, without trend data. The process 200 begins at a start step, and then moves to step 202, wherein the engine 120 collects demand data 112. The engine 120 can retrieve demand data 112 from the database 110. At step 204, the engine 120 collects supplier data 114, such as source lead time data. The engine 120 can retrieve supplier data 114 from the database 110. In some embodiments, the demand module 122 can collect demand data 112 and the supplier lead time module 124 can collect supplier data 114. The engine 120 can collect demand data 112 and supplier data 114 simultaneously or separately, in any order.

At step 206, the engine 120 can calculate the safety stock recommendation. The mean and standard deviation of the demand data 112 can be used to calculate the safety stock recommendation. In some embodiments, the mean and standard deviation of the demand data 112 are calculated for the last twelve months. The mean and standard deviation of the supplier data 114 can be used to calculate the safety stock recommendation. In some embodiments, the mean and standard deviation of the supplier data 114 are calculated for the last twelve months. In some embodiments, the engine 120 can calculate the safety stock recommendation. At step 208, the engine 120 can output the safety stock recommendation in units and days. The engine 120 can output the recommendation as a quantity of units or as a measure of time, such as days. In some embodiments, the analytic module 126 can output the safety stock recommendation to the user interface 130.

At step 210, the engine 120 can generate demand and supplier graphs and then output those graphs to the user interface 130. In some embodiments, the analytic module 126 can generate graphs for the user interface 130. In some embodiments, the demand data 112 is used to generate at least one demand graph and the supplier data 114 is used to generate at least one supplier graph. The engine 120 can generate scatter plots as described herein. The engine 120 can generate scatter plots with both included and exclude data points represented as different icons. The engine 120 can generate at least one bar graph to illustrate the supply and the demand contributions to the safety stock recommendation.

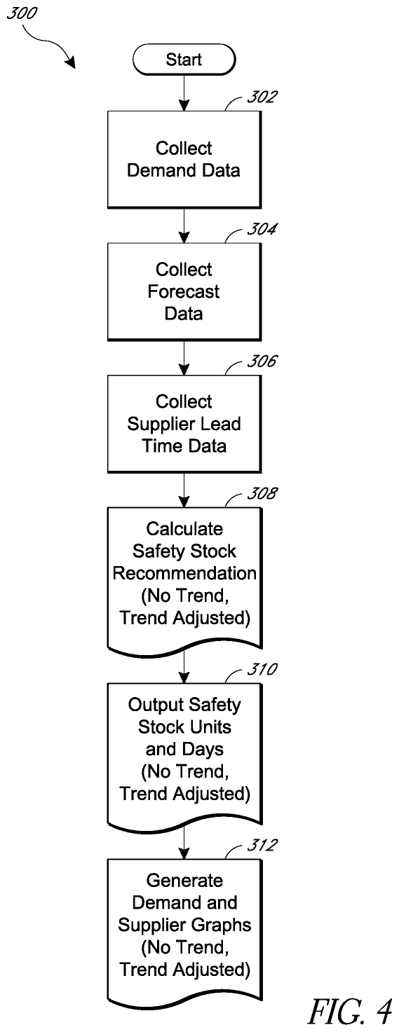

FIG. 4 is a flowchart illustrating an example process 300 of the engine 120 of the SSM system 100, with trend or forecast data. The process 300 can include any of the steps of example process 200 or any other method described herein. The process 300 begins at a start step, and then moves to step 302, wherein the engine 120 collects demand data 112. The engine 120 can retrieve demand data 112 from the database 110. At step 304, the engine 120 collects forecast data related to demand. The engine 120 can retrieve forecast data from the database 110. The engine can model the demand data 112 to prepare forecast data. At step 306, the engine 120 collects supplier data 114. The engine 120 can retrieve supplier data 114 from the database 110. In some embodiments, the demand module 122 can collect demand data 112. In some embodiments, the supplier lead time module 124 can collect supplier data 114. The engine 120 can collect demand data 112 and supplier data 114 simultaneously or separately, in any order.

At step 308, the engine 120 can calculate the safety stock recommendation without utilizing the forecast data, indicated in FIG. 4 as “No Trend”. The mean and standard deviation of the demand data 112 can be used to calculate the safety stock recommendation. In some embodiments, the mean and standard deviation of the demand data 112 can be calculated for the last twelve months. The mean and standard deviation of the supplier data 114 can be used to calculate the safety stock recommendation. In some embodiments, the mean and standard deviation of the supplier data 114 can be calculated for the last twelve months. In some embodiments, the analytic module 126 can calculate the safety stock recommendation without trend data.

At step 308, the engine 120 can calculate the safety stock recommendation utilizing the forecast data, indicated in FIG. 4 as “Trend Adjusted”. The mean and standard deviation of the demand data 112 can be used to calculate the safety stock recommendation. The mean of the demand data 112 can be the projected mean for the next month. The standard deviation of the demand data 112 can be calculated after adjusting for the trend. The mean and standard deviation of the supplier data 114 can be used to calculate the safety stock recommendation. In some embodiments, the mean and standard deviation of the supplier data 114 are calculated for the last twelve months. In some embodiments, the analytic module 126 can calculate the safety stock recommendation with trend data.

At step 310, the engine 120 can output the safety stock recommendation in units and days. The engine 120 can provide an output with adjusting for the trend in demand data 112 and without adjusting for the trend in demand data 112. The engine 120 can output the recommendation as a quantity of units and/or as a measure of time, such as days. In some embodiments, the analytic module 126 can output the safety stock recommendation to the user interface 130.

At step 312, the engine 120 can generate demand and supplier graphs. In some embodiments, the analytic module 126 can generate graphs for the user interface 130. In some embodiments, the demand data 112 can be used to generate at least one demand graph and the supplier data 114 can be used to generate at least one supplier graph. In some embodiments, the forecast data can be used to generate at least one demand graph. In some embodiments, the demand data 112 can be used to generate at least one demand graph with the forecast data. In some embodiments, the demand data 112 can be used to generate at least one demand graph without the forecast data.

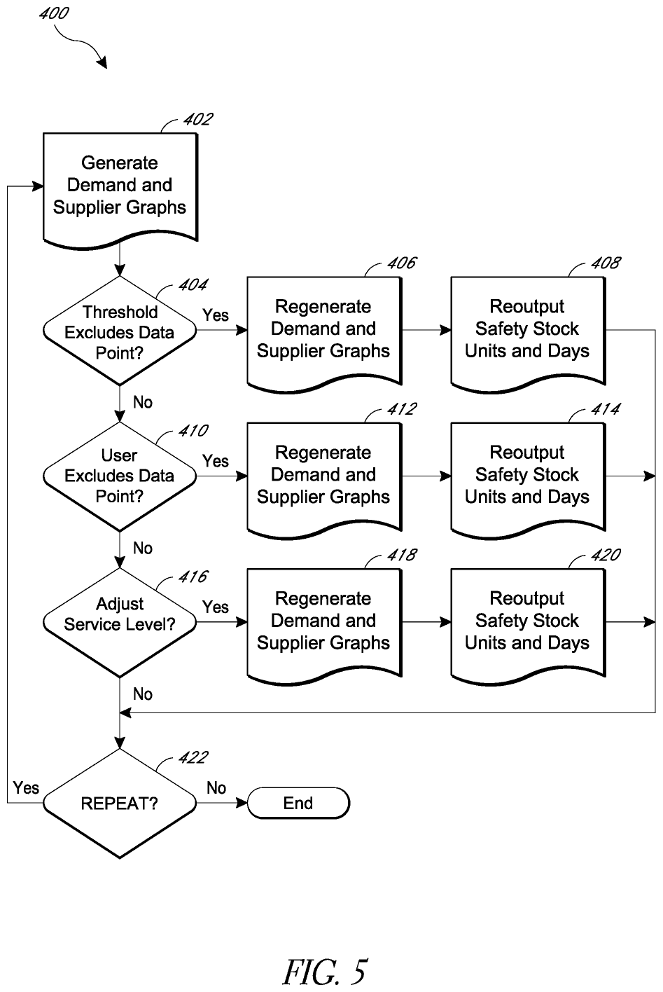

FIG. 5 is a flowchart illustrating an example process 400 of the engine 120 of the SSM system 100 with user adjustments. The process 400 can include any of the steps of example process 200, example process 300, or any other method described herein. The process 400 begins at step 402 wherein the engine 120 can generate demand and supplier graphs.

At decision step 404, the user can exclude one or more data points based on a threshold. The engine 120 can calculate the demand threshold for the demand data 112. The engine 120 can calculate the supply threshold for the supplier data 114. The user can interact with the user interface 130 to exclude data outside of the demand threshold, for instance exclude data above a demand ceiling or exclude data below a demand floor. The user can interact with the user interface 130 to exclude data outside of the supply threshold, for instance exclude data above a supply ceiling or exclude data below a supply floor. The user can interact with a physical input, such as a button, or a digital input, such as selecting an icon to exclude data outside of the demand threshold or the supply threshold. If the user excludes one or more data points based on the demand threshold, the supply threshold, or both the demand and supply thresholds, then the engine 120 regenerates demand and supplier graphs based on the included data at step 406. If the user excludes data points based on the demand threshold, the supply threshold, or both the demand and supply thresholds, then the engine 120 re-outputs the safety stock recommendation at step 408. If the user does not exclude data points based on the threshold, the process 400 continues to step 410.

At decision step 410, the user can exclude one or more data points based on a user's selection. The interactive graphical display 132 can allow the user to see one or more data points of the demand and supplier graphs. The user can select one or more data points. The user can interact with a physical input on the user interface 130, such as a button, or interact with a digital input, such as selecting a point by or hovering over a point. The user can select more than one point, such as by drawing a perimeter around two or more points, clicking on two or more points, or drawing a line separating a plurality of points. The user can exclude data points on the demand graph, the supply graph or both the demand and supply graphs. If the user excludes data points based on user selection, then the engine 120 regenerates demand and supplier graphs based on the included data at step 412. If the user excludes data points based on user selection, then the engine 120 re-outputs the safety stock recommendation at step 414. If the user does not exclude data points based on the user's selection, the process 400 continues to step 416.

At decision step 416, the user can adjust the service level. Adjusting the service level is one example of a user input, but other inputs are contemplated as discussed herein. The interactive graphical display 132 can allow the user to see options for one or more inputs. The user can interact with a physical input on the user interface 130 to select an input, such as a typing a service level, or interact with a digital input, such sliding an icon along a scale. If the user changes the service level, then the engine 120 regenerates demand and supplier graphs based on the service level at step 418. If the user changes the service level, then the engine 120 re-outputs the safety stock recommendation at step 420. If the user does not change the service level, the process 400 continues to step 422. At step 422, the process 400 makes a determination if it will repeat. If a determination is made that the process will not repeat, then the process 400 moves to an end state. If a determination is made that the process 400 should repeat, then it returns to the state 402 to generate demand and supplier graphs.

The process 400 can repeat by the user excluding data related to a threshold. The process 400 can repeat by the user excluding data by selecting one or more data points. The process 400 can repeat by the user adjusting an input, such as the service level. In some embodiments, after each change by the user, the engine 120 regenerates demand and supplier graphs. The engine 120 can regenerate demand and supplier graphs in real-time, or approximately real-time based on processing speeds, upon manipulation by the user. In some embodiments, after each change by the user, the engine 120 re-outputs the safety stock recommendation. The engine 120 re-outputs the safety stock recommendation in real-time, or approximately real-time, upon manipulation by the user.

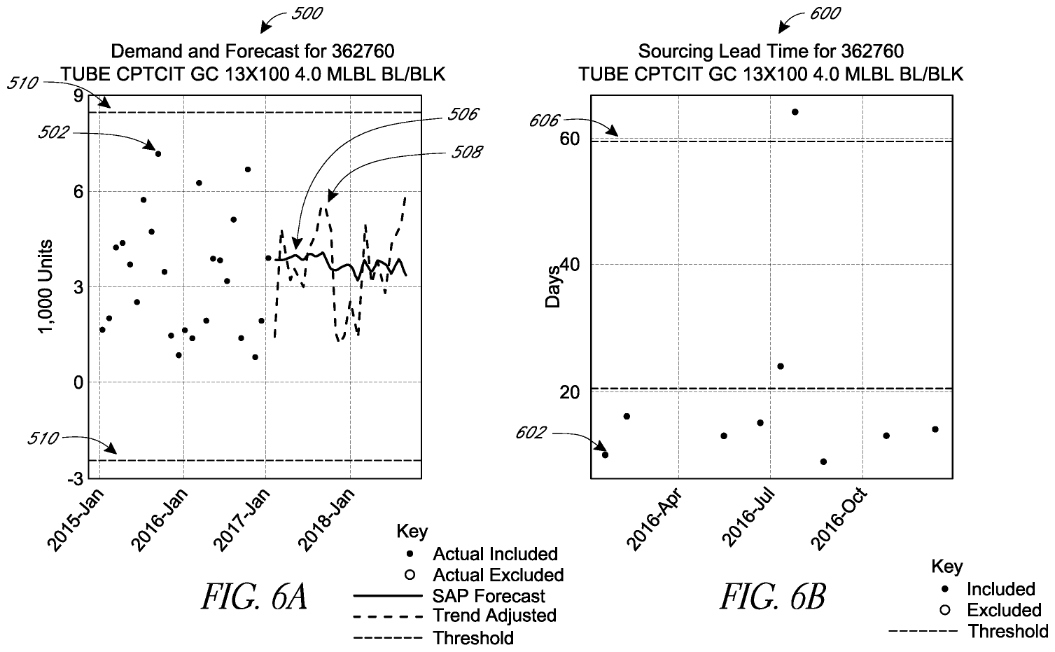

FIG. 6A is a graph illustrating an example demand graph 500 of the interactive graphical display 132. The demand graph 500 illustrates the demand data 112, or a portion thereof. The demand graph 500 can be a scatter plot in a demand coordinate system. The demand coordinate system can include two variables, with units along the y-axis and date along the x-axis. The demand graph 500 can illustrate number of units per unit of time. In the illustrated example, the demand graph 500 can display data points for January 2015 to January 2017. In the illustrated example, the demand graph 500 can include a forecast for the demand data after January 2017, for instance beyond January 2018.

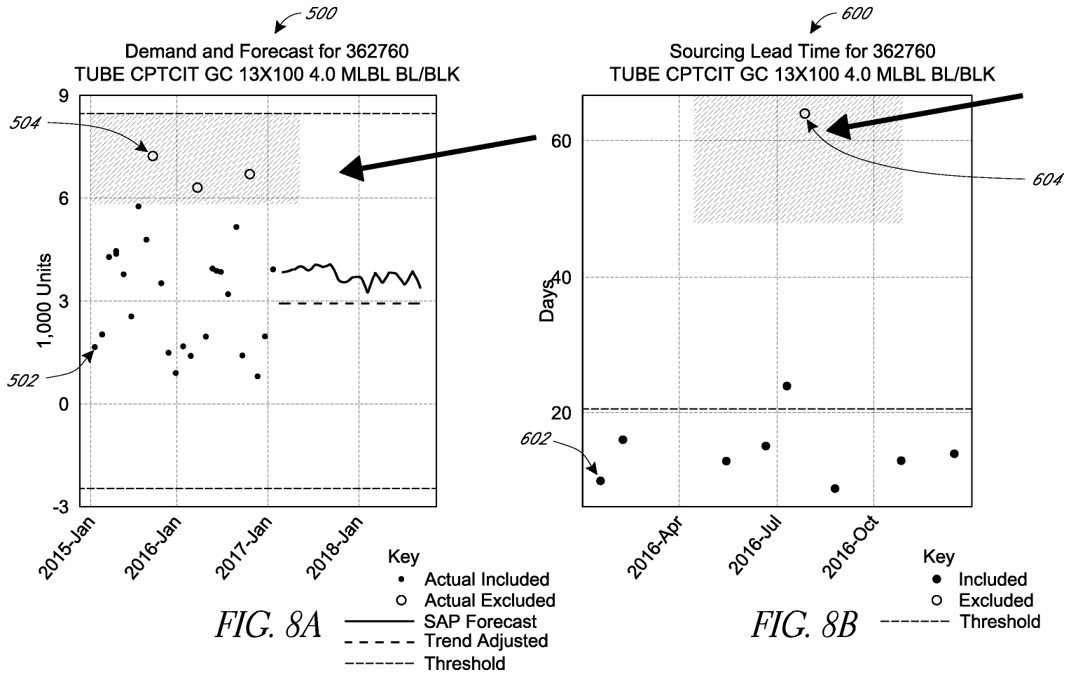

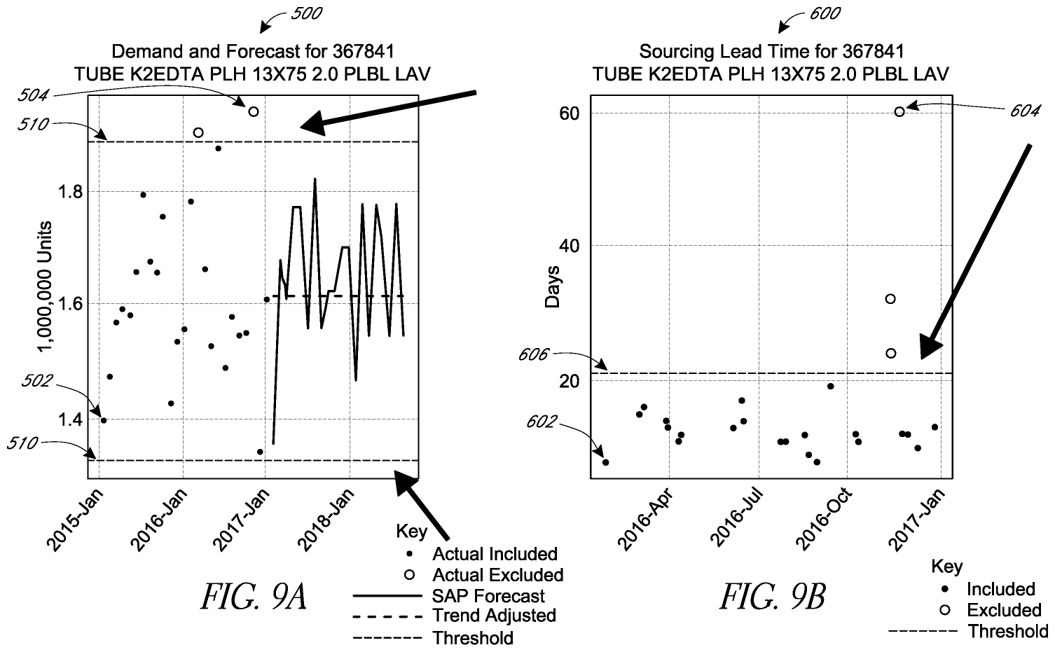

The demand graph 500 can illustrate included data points 502 with an icon. In the illustrated embodiment, the included data points 502 are shown as a filled or solid dot. The demand graph 500 can illustrate excluded data points 504 with an icon, see FIGS. 8A and 9A. The excluded data points 504 can be shown as a circle. Other icons are contemplated to distinguish include and excluded data points.

The demand graph 500 can illustrate the forecast 506. The forecast can be a trend in the demand data 112 to predict future demand. As one example, the demand data 112 can have a seasonal or cyclic trend. As another example, the demand data 112 can have an upward trend. The demand graph 500 can illustrate the forecast 506 with an icon. In the illustrated embodiment, the forecast 506 is shown as a solid line. The forecast data can be utilized to generate trend adjusted, predicted data points. The demand graph 500 illustrates trend adjusted, predicted data points 508 with an icon. In the illustrated embodiment, the trend adjusted, predicted data points 508 are shown as a dashed line.

The demand graph 500 can illustrate the threshold 510 with an icon. The demand graph 500 can include an upper threshold and a lower threshold, which can have the same or different icons. In the illustrated embodiment, the thresholds 510 are shown as a faint dashed line.

FIG. 6B is a graph illustrating an example supplier graph 600 of the interactive graphical display 132. The supplier graph 600 can illustrate the supplier graph 614, or a portion thereof. The supplier graph 600 can be a scatter plot in a source lead time coordinate system. The supplier graph 600 can illustrate number of days of sourcing lead time per unit of time. In the illustrated example, the supplier graph 600 can display data points for April 2016 to October 2016. The supplier graph 600 can illustrate included data points 602 with an icon. In the illustrated embodiment, the included data points 602 are shown as a filled or solid dot. The supplier graph 600 can illustrate excluded data points 604 with an icon, see FIGS. 8B and 9B. The excluded data points 604 can be shown as a circle. Other icons are contemplated to distinguish include and excluded data points. The supplier graph 600 illustrates a threshold 606 with an icon. The supplier graph 600 can include an upper threshold. In the illustrated embodiment, the threshold 606 is shown as a faint dashed line.

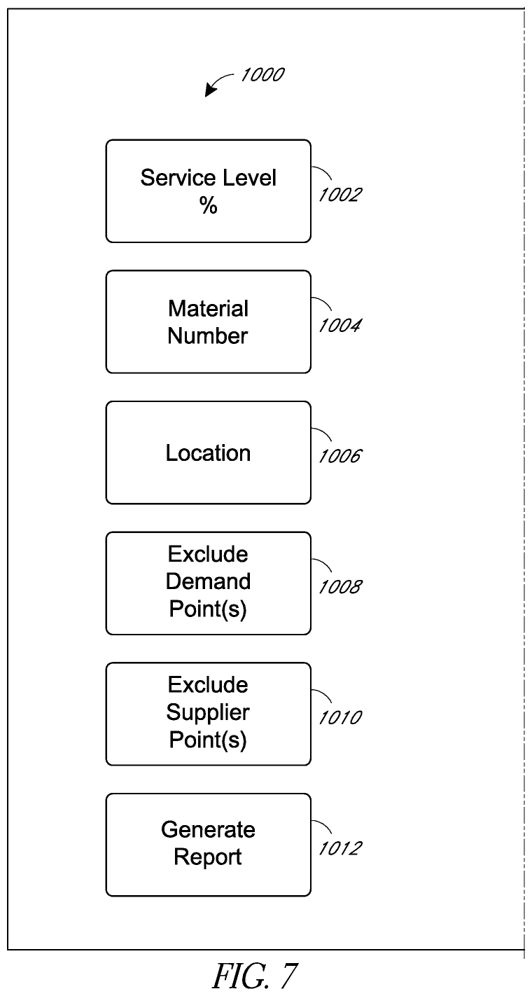

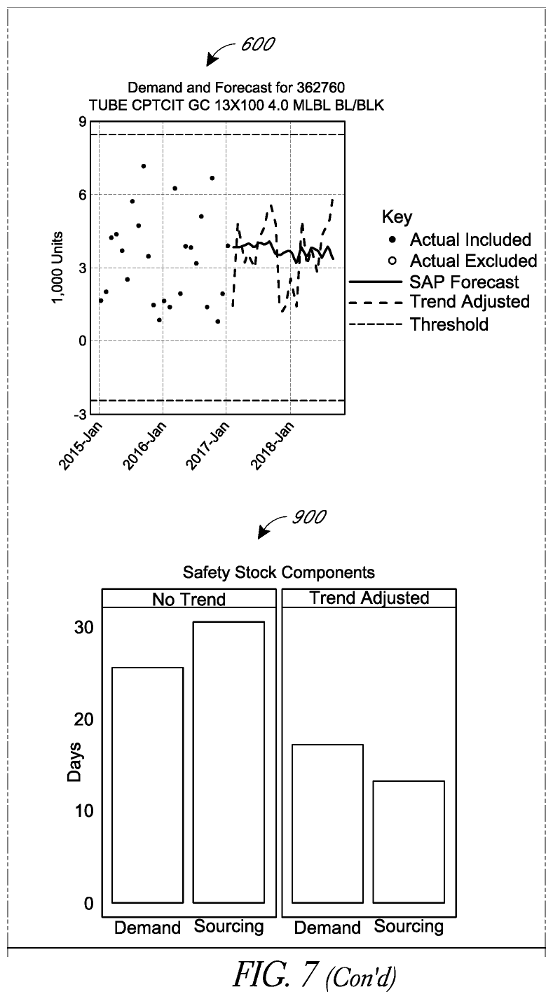

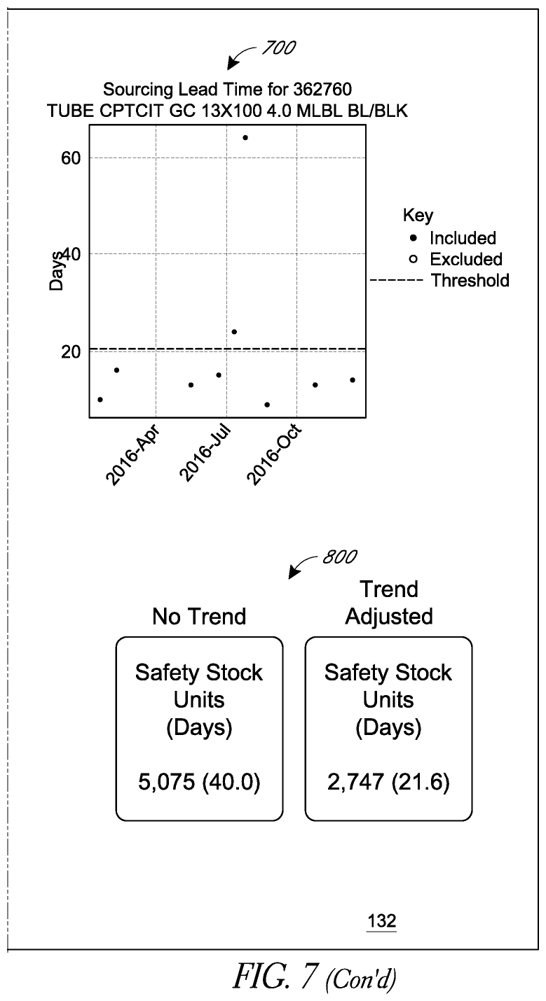

FIG. 7 is an example of the interactive graphical display 132. The interactive graphical display 132 can present the demand graph 600 and the supplier graph 700 described herein. The demand graph 600 and the supplier graph 700 can be displayed a parallel or side-by-side orientation. The user can visualize both graphs on the interactive graphical display 132. While scatter plots are shown, other types of graphs are contemplated for the demand graph 600 and the supplier graph 700.

The interactive graphical display 132 can display the safety stock recommendation 800. The safety stock recommendation 800 can be calculated and presented as a number of units and a number of days. The safety stock recommendation 800 can include an output not utilizing the forecast (e.g., “No Trend”) and an output utilizing the forecast (e.g., “Trend Adjusted”).

The interactive graphical display 132 can present a graph of safety stock components 900. The graph of safety stock components 900 can illustrate the contribution of each of the demand data 112 and the supplier data 114 to the safety stock calculation. The graph of safety stock components 900 can illustrate the contribution of each of the demand data 112 and the supplier data 114 with and without the trend. The graph of safety stock components 900 can illustrate the number of days of the safety stock recommendation are due to the demand and the supplier. While bar graphs are shown, other types of graphs are contemplated for the graph of safety stock components 900.

The interactive graphical display 132 can include one or more inputs 1000 designed to be manipulated by the user. The input 1002 relates to the service level. The user can change the service level to change the safety stock recommendation 800. The user can enter a number, such as 96, to indicate the desired service level. The user can automate the model settings. The input 1004 relates to the material number. The user can enter the material number to select the product. In some embodiments, the material number relates to a medical product. The material number can be a part number or other identifier. The material number can be designated by the company. The input 1006 relates to the location. In some embodiments, the user can select a plant from one or more manufacturing plants. In some embodiments, the user can select a distribution center from one or more distribution centers. The input 1008 relates to threshold excluded data points for demand data 112. The user can select whether to exclude values outside of the threshold for demand data 112. In some embodiments, the user can select whether to exclude values outside of the threshold for each demand threshold. The input 1010 relates to threshold excluded data points for supplier data 114. The user can select whether to exclude values outside of the threshold for supplier data 114. The input 1012 relates to generating a report. The user can select whether to generate a report based on the data displayed on the interactive graphical display 132. The report can include the demand data 112 and the supplier data 114, or a selected portion thereof. The report can include the safety stock recommendation 800. The report can include any data described herein.

FIG. 8A is a graph illustrating an example demand graph 500 with user excluded data. FIG. 8B is a graph illustrating an example supplier graph 600 with user excluded data. During interactive use, the user can select one or more data points 504, 604 to be excluded. The included data points 502, 602 are also illustrated. As one non-limiting example, a box can be drawn on the demand graph 500, the supply graph 600, or both the demand and supply graphs. FIG. 8A illustrates a box with three excluded demand data points 504. The user can exclude all data points within the box. The user can redraw the box. In some embodiments, the user can have more control over what data points are excluded as compared with the calculated threshold. In some embodiments, the user can have more flexibility over what data points are excluded. FIG. 8B illustrates a box with one excluded supplier data point 604. In some embodiments, the user can select individual data points to exclude. The excluded data point selected by the user can be any data point on the demand and supply graphs. The excluded data point 504, 604 may not have a common perimeter or be outside a single line.

FIG. 9A is a graph illustrating an example demand graph with threshold excluded data points. FIG. 9B is a graph illustrating an example supplier graph with threshold excluded data points. One or more data points 504, 604 can be excluded. The included data points 502, 602 are also illustrated. As described herein, the engine 120 can calculate one or more thresholds related to the demand data 112 and the supplier data 114. The demand data 112 or the supplier data 114 can be skewed such that one or more data points lie above or below the threshold. Typically, most data cluster together, but occasionally there can be an outlier, such as a much higher or lower data point. For some cases, these outliers are representative of the process and can be included in the calculations for the safety stock recommendation. For other cases, these outliers are clear exceptions and the user can remove such outliers from the safety stock recommendations. One non-limiting example of an outlier is a Tender order. In some embodiments, the user can analyze the demand and the supplier graphs and the safety stock recommendation by including and excluding outliers to understand the impact of the outliers.

As described herein, thresholds can be used to define extreme values. In some embodiments, for the demand data 112, there can be a threshold 510 for low and high extreme values. FIG. 9A illustrates the threshold 510 for low and high extreme values. In some embodiments, for the supplier data, there can be a threshold 606 for high extreme values. FIG. 9B illustrates the threshold 606 for high extreme values. Other numbers and orientations of threshold can be available for the user to select.

In some embodiments, during interactive use of the user interface 130, the demand and supplier data can be trimmed of extreme values independently. In some embodiments, during interactive use of the user interface 130, the high values and the low values of the demand data 112 can be trimmed independently. In some embodiments, during interactive use of the user interface 130, the high values of the demand data 112 and the high values of the supplier data 114 can be trimmed independently. In some embodiments, during interactive use of the user interface 130, the demand and supplier data can be trimmed of extreme values simultaneously. In some embodiments, during interactive use of the user interface 130, the high values and the low values of the demand data 112 can be trimmed simultaneously. In some embodiments, during interactive use of the user interface 130, the high values of the demand data 112 and the high values of the supplier data 114 can be trimmed simultaneously.

In some embodiments, the engine 120 can utilize an algorithm to calculate extreme value thresholds 510, 606. The engine 120 can calculate the extreme values from a formula adapted from boxplots. As one non-limiting example, the low threshold can be the 25 percentile minus 1.5 times the inner quartile range. As one non-limiting example, the high threshold can be the 75 percentile plus 1.5 times the inner quartile range. In some embodiments, the thresholds are calculated once using all available data.

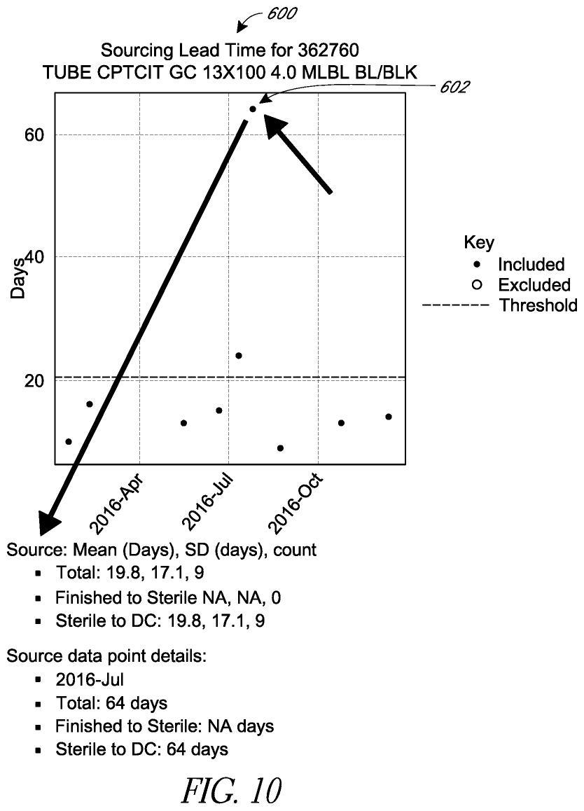

FIG. 10 is a graph illustrating an example supplier graph 600 with a selected data point 602. During interactive use, the user can select a data point in order to receive more information regarding the data point. The interactive graphical display 132 can present the additional information for the selected point 602. The additional information can include the date and the number of days for a selected point on the supplier graph 600. The additional information can include the days to sterilization and the days from sterilization to the distribution center for a selected point on the supplier graph. While FIG. 10 illustrates the supplier graph 600, a data point can be selected on the demand graph 500 or the supplier graph 600. The additional information can include the date and the number of units for a selected point on the demand graph (not shown). The additional information can include name or location of the distribution center for a selected point on the demand graph (not shown). The additional information can include name or location of the end user for a selected point on the demand graph (not shown).

The user can utilize the additional information to understand outliers of the demand graph 500 or the supplier graph 600. The user can better understand the impact of the outliers on the safety stock recommendation. In some embodiments, the user can utilize the additional information to sort out delays on the supplier side. In some embodiments, the user can utilize the additional information to understand demand trends. Other uses of the data are contemplated.

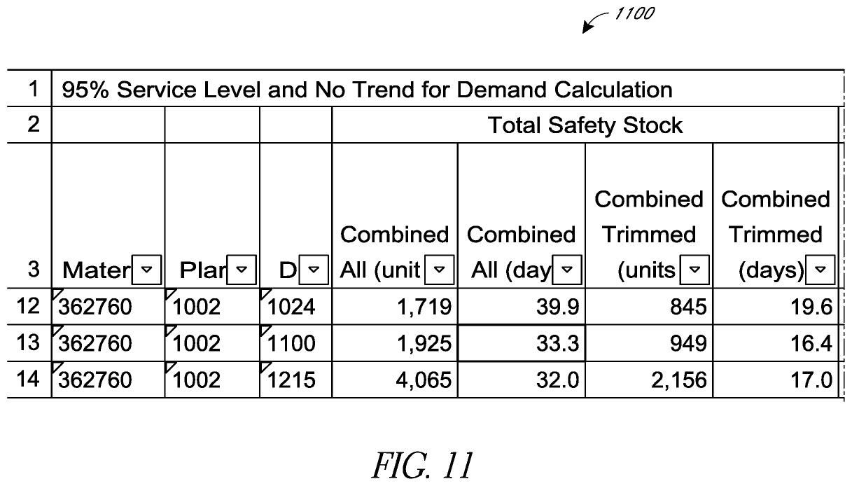

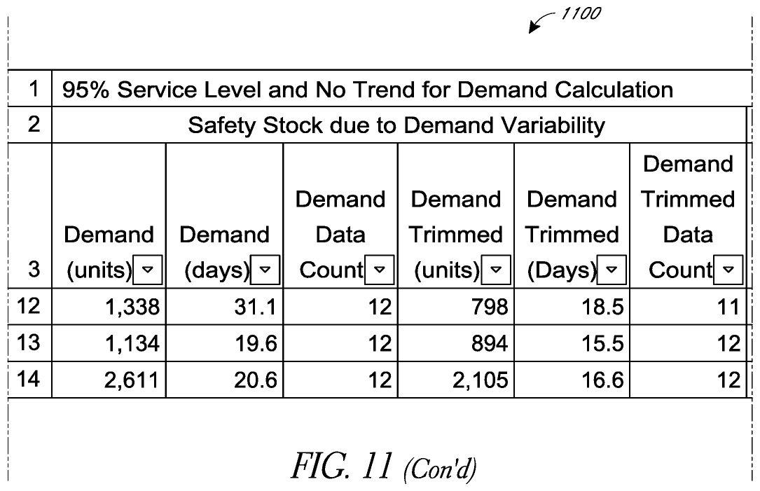

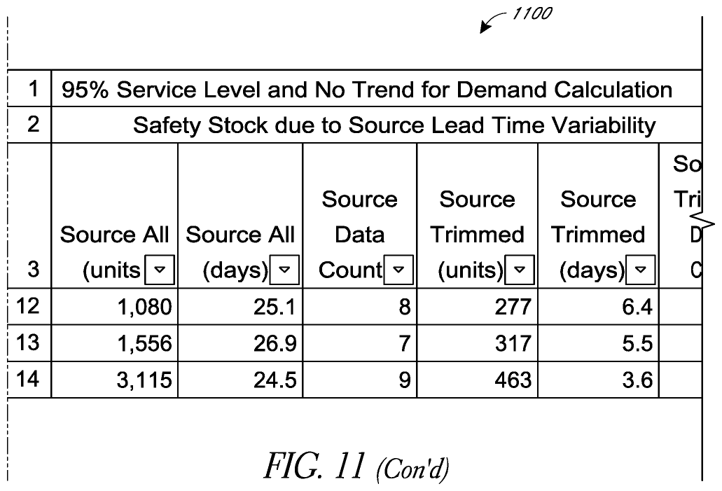

FIG. 11 is an example of a report 1100. The report can be generated by the user at any time. The report 1100 can be generated before or after manipulating the data points or one or more inputs. The report 1100 can provide the demand data 112 and the supplier data 114 utilized to provide the safety stock recommendation, e.g., the included data points. The report 1100 can provide the demand data 112 and the supplier data 114 that was excluded, either by the one or more thresholds or by the user. The report 1100 can be in any format including a spreadsheet. In some embodiments, the report 1100 can be stored by the SSM system 100 as described herein. The graphs shown in FIGS. 6A-10 and the report shown in FIG. 11 are provided by way of example only and are not intended to be limiting.

As described herein, the mean and the standard deviation are estimated for the demand data 112 and the supplier data 114. There can be various methods to estimate the mean and standard deviation. The estimates can be combined to create a safety stock recommendation. The formulas below can allow the SSM system to combine estimates for the safety stock recommendation. The SSM system can utilize one or more of the example formulas described herein. The example formulas presented herein can be modified for coding efficiency. The example formulas presented herein can represent how the calculations are performed by the SSM system. The output of the SSM system can be verified by performing one or more simulations to confirm the implemented formulas are correct.

The example formula to calculate safety stock due to demand variability is represented below. The Z_{service level} is the Z-score appropriate for the service level. The Z-score for the default service level, 98%, is 2.05. SD_{S} is the standard deviation of the demand. L is the average of the lead time. T is a factor to adjust for differences in the lead time period and the demand period. If both periods are in months, then this factor is 1.

SS_{D}=Z_{service level}SD\sqrt{L/T} (1)

The example formula to calculate safety stock due to lead time variability is represented below. The Z_{service level} is the Z-score appropriate for the service level. The Z-score for the default service level, 98%, is 2.05. SD_{L} is the standard deviation of the lead time. D is the average of the demand.

SS_{L}=Z_{service level}SD_{L}D̅ (2)

In some embodiments, the variability due to demand and lead time are independent. In some embodiments, the SSM system can assume the variability in demand data and supplier data are independent. The example formula to combine the two components and calculate the total safety stock is represented below.

SS_{Total}=\sqrt{SS_{D}²+SS_{L}²} (3)

The example formula to convert units to days is represented below. The formulas for safety stock are expressed in units, or amount of product. To express this in demand days the following conversion can be used. SS_{Days} is safety stock expressed in days. SS_{Units} is safety stock expressed in units. D is the expected demand in units per month. D can be estimated with the average forecasted demand for the next 3 months.

$${SS}_{Days} = \frac{365.25 Days}{12 Months} \frac{{SS}_{Units}}{\overline{D}} \qquad ( 4 )$$

In some embodiments, the forecast demand came from the business software, e.g., the SAP® Forecast, for the “No Trend” calculation and the model for the “Trend Adjusted” calculation. In some embodiments, the forecast demand came from the business software, e.g., the SAP® Forecast, for the “No Trend” calculation and “Trend Adjusted” calculation.

Implementations disclosed herein provide systems and methods for SSM systems. One skilled in the art will recognize that these embodiments may be implemented in hardware, software, firmware, or any combination thereof.

The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. Disk and disc, as used herein, includes compact disc (CD), laser disc, optical disc, digital versatile disc (DVD), floppy disk and Blu-ray® disc where disks usually reproduce data magnetically, while discs reproduce data optically with lasers. It should be noted that a computer-readable medium may be tangible and non-transitory. The term “computer-program product” refers to a computing device or processor in combination with code or instructions (e.g., a “program”) that may be executed, processed or computed by the computing device or processor. As used herein, the term “code” may refer to software, instructions, code or data that is/are executable by a computing device or processor.

Software or instructions may also be transmitted over a transmission medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of transmission medium.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like. The phrase “based on” does not mean “based only on,” unless expressly specified otherwise. In other words, the phrase “based on” describes both “based only on” and “based at least on.”

In the foregoing description, specific details are given to provide a thorough understanding of the examples. However, it will be understood by one of ordinary skill in the art that the examples may be practiced without these specific details. For example, electrical components/devices may be shown in block diagrams in order not to obscure the examples in unnecessary detail. In other instances, such components, other structures and techniques may be shown in detail to further explain the examples.

It is also noted that the examples may be described as a process, which is depicted as a flowchart, a flow diagram, a finite state diagram, a structure diagram, or a block diagram. Although a flowchart may describe the operations as a sequential process, many of the operations can be performed in parallel, or concurrently, and the process can be repeated. In addition, the order of the operations may be re-arranged. A process is terminated when its operations are completed. A process may correspond to a method, a function, a procedure, a subroutine, a subprogram, etc. When a process corresponds to a software function, its termination corresponds to a return of the function to the calling function or the main function.

The previous description of the disclosed implementations is provided to enable any person skilled in the art to make or use the present invention. Various modifications to these implementations will be readily apparent to those skilled in the art, and the generic principles defined herein may be applied to other implementations without departing from the spirit or scope of the invention. Thus, the present invention is not intended to be limited to the implementations shown herein but is to be accorded the widest scope consistent with the principles and novel features disclosed herein.

1. A system for providing safety stock modeling for a medical product, the system comprising:
a database of information related to a plurality of medical products;
an engine comprising a demand module, a sourcing lead time module, and at least one analytic module for calculating a trend in data points from the database of information; and
an user interface configured to allow the user to manipulate an interactive graphical display, wherein the interactive graphical display illustrates data points from the database of information related to a single product in a demand coordinate system and a sourcing lead time coordinate system,
wherein the engine is configured to output an optimal safety stock recommendation based on included data points in the demand coordinate system and included data points in the sourcing lead time coordinate system, wherein the engine is configured to update the optimal safety stock recommendation as the user changes the included data points.

2. The system of claim 1, wherein the database of information comprises data over at least two years.

3. The system of claim 1, wherein the engine is configured adjust the optimal safety stock recommendation based on the trend calculated by the at least one analytic module.

4. The system of claim 1, wherein the trend is a cyclic demand.

5. The system of claim 1, wherein the interactive graphical display is updated in real-time when the user changes the included data points.

6. The system of claim 1, wherein the interactive graphical display provides information from the database of information on a selected data point.

7. The system of claim 1, wherein the engine is configured to provide a demand threshold in the demand coordinate system and a sourcing threshold in the sourcing lead time coordinate system.

8. The system of claim 7, wherein the optimal safety stock recommendation is updated when the user excludes one or more data points above the demand threshold or the sourcing threshold.

9. The system of claim 7, wherein the optimal safety stock recommendation is updated when the engine excludes one or more data points above the demand threshold or the sourcing threshold.

10. The system of claim 1, wherein the engine is configured to create a report from the included data points.

11. The system of claim 1, wherein the optimal safety stock recommendation is updated when the service level is adjusted.

12. The system of claim 1, wherein the interactive graphical display is configured to allow the user to visualized data points in the demand coordinate system and the sourcing lead time coordinate system simultaneously.

13. The system of claim 1, wherein the user interface is configured to allow the user to interactively exclude data points in the demand coordinate system and the sourcing lead time coordinate system simultaneously.

14. The system of claim 1, wherein the user interface is configured to allow the user to interactively exclude data points in the demand coordinate system and the sourcing lead time coordinate system independently.

15. The system of claim 1, wherein the engine is configured to output the optimal safety stock recommendation based on the mean and standard deviation of the included data points.

16. The system of claim 1, wherein the engine is configured to output an optimal safety stock recommendation for a family of related medical products.

17. A method for providing safety stock modeling for a medical product, the method comprising:
storing information related to a plurality of medical products in a database;
generating with an engine an interactive graphical display illustrating data points related to demand and sourcing lead time; and
updating, in real time, the interactive graphical display and an optimal safety stock recommendation by adjusting an input selected from the group consisting of a trend of data points, a user excluded data point, a user included data point, and an engine excluded data point.

18. The method of claim 17, wherein the input is a user excluded data point, wherein the user excluded data point is above a demand threshold or a sourcing threshold.

19. The method of claim 17, wherein the input is an engine excluded data point, wherein the engine excluded data point is above a demand threshold or a sourcing threshold.

20. The method of claim 17, further comprising updating, in real time, the interactive graphical display and the optimal safety stock recommendation based on service level.
